(19) United States
(12) Patent Application Publication
(10) Pub. No.: US 2018/0132635 A1
(43) Pub. Date: May 17, 2018

(54) Smart Pillow

(71) Applicant: Rick Loos, Del Mar, CA (US)

(72) Inventor: Rick Loos, Del Mar, CA (US)

(21) Appl. No.: 15/320,221

(22) PCT Filed: Jun. 19, 2015
(86) PCT No.: PCT/US2015/036782

§ 371 Date: Dec. 19, 2016

(51) Int. Cl. A47G 9/10; A61M 21/00

(52) U.S. Cl. CPC A47G 9/1045; A61M 21/00; A47G 2200/146; A47G 2200/166; A61M 2230/63; A61M 2021/0027; A61M 2021/0083; A61M 2230/42; A61M 2230/50; A47G 2200/20

(57) ABSTRACT

A pillow is provided which is configured with sensors in communication with a controller having a computing component and software running in electronic memory. Using input data streams from the sensors to the controller, the software running on the computing component can ascertain a current sleep level of a user as being in a REM cycle or non REM cycle, The controller is configured to override a user set alarm time on an alarm, with a new activation time should it be determined the user will be in a REM cycle at the user chosen time for the alarm.

This application claims priority to U.S. Provisional Patent Application Ser. No. 62/014,585 filed on Jun. 19, 2014 which is incorporated herein in its entirety by this reference thereto.

### BACKGROUND OF THE INVENTION

### 1. Field of the Invention

The present invention relates to pillows and sleeping using such. More particularly, it relates to a pillow configured with a plurality of sensors in a plurality of zones which are adapted to sense and communicate data concerning physiological aspects of the user during the user's duration of sleep and current state of sleep. Such data is reviewable from electronic memory subsequently or in real time for discerning aspects of the user's current health, and may be employed with software to act as an adaptive alarm to wake the user during an opportune window of a sleep cycle.

### 2. Prior Art

Conventionally, pillows consist of a top and bottom surface sewn together at the perimeter edges to form an interior cavity which holds stuffing or padding. This single sewn seam central engagement structurally causes the top and bottom surfaces to slope alongside edges toward the circumferentially located seam. The slope is somewhat of an arc from the widest point of the pillow at a mid section toward the seam from the top and bottom surfaces.

Such pillows are employed by most sleepers who use a bed in Western society and therefor are constantly positioned in a bed, in close contact with the head of a user or sleeper while the user is sleeping or awakening. Consequently, unlike other portions of the bed where portions of the user's body will occupy for short periods and then move elsewhere or change position, the user's head contact with a pillow is constant. This constant contact particularly includes three sides of the head which might be in contact with any of three areas of the pillow, which using sensors can correlate to the user's body position at the time.

However, most such art concerning pillows is related to providing shapes, contours, sizes, and recesses to make the user comfortable. For instance, many pillows provide a ridge for the neck of a sleeper and depression for the head such that the neck of the user is supported in a straight position. There are other pillows which provide edge-located voids for the user's shoulders to properly position the side portions of the pillow for side sleepers. There are also pillows made from foam, oats, rice, down, and a plethora of other substances with varying claims for the user's health and sleeping posture.

To date however, there are no pillows which are adapted for collecting real time data on a user or sleeper through the employment of pillow-mounted sensors in multiple zones, which provide real time data employable by software running on a computing device, to help both plan user sleep, monitor their initial stages of sleep for problems, and provide a means to wake them at a calculated time, which is most opportune for alertness.

While there are pillows for temperature and pillows for other purposes, there is no pillow in the prior art which employs a plurality of sensors, in multiple segments of a pillow, which are adapted to provide electronic data streams therefrom, employable by software on a computing device to determine user position and health and bodily functions. Further, there is no prior art wherein multiple data streams, from multiple types of pillow-located sensors, in multiple pillow areas, can be collected and provided to the user or medical professionals to sense certain health issues which may display symptoms during sleep and to provide data concerning long term sleep.

As such, there exists an unmet need, for a pillow which includes components operatively engaged and configured, to survive the rigors of a sleeping user over time. Such a pillow device should position one or preferably a plurality of sensors adjacent to the user's head, in predicted positions or areas upon the pillow, to capture and electronically communicate data concerning multiple physiological aspects of the user, and determine the user's current sleeping state, as well as data concerning general and specific health issues. Such a device and method should include a plurality of such sensors, each for sensing different data correlating to body physiological conditions or actions or positions of the user during sleep.

Such a system should be configured electronically communicate the gathered data to a computing device using wired or wireless communication, in real time for storage in electronic memory. Using the data from the sensors in memory, software running on the device, which is adapted to employ the plurality of different data streams, can determine the most likely current state of user sleep. Further, such software may be adapted to correlate electronically stored and collected data from the sleeping user, with potential health issues exhibited during sleep that can be determined from the data communicated from the sensors. For example cessation of breathing can be sensed by a microphone as can gasping subsequent to such a cessation. The timing of the length of cessation can be employed to ascertain sleep apnea using a database of stored information concerning durations attributed to such.

Further, such a system can employ software or electronic communication means to store in memory and communicate captured sensor data, which may be further employed by medical professionals and programs adapted to report to the user their sleeping habits and health concerns over single nights and long periods of time.

Finally, in addition to employing the data gathered from the head and neck area which are substantially always in contact with a pillow, to discern health issues, the system should also employ software adapted to use the real time streaming data from the multiple sensors, to discern a current sleeping state of the user, and using software adapted to calculate an appropriate waking time to activate an appropriate means to wake the user at the time calculated as most beneficial for wakefulness and alertness during the coming day.

### SUMMARY OF THE INVENTION

The device, herein disclosed and described, achieves the above-mentioned objects and goals, through the provision of a pillow which includes a plurality of sensing components operatively in position within the pillow, to detect and communicate individual data streams representative of individual body actions, conditions, positions, and other sensed data. These individual streams of data concerning detected physical information, temperature, sound, or other ongoing changes and actions can be employed and the data retrieved communicated to software adapted to the task and running on a computing device, to provide the user with discerned health information and to calculate sleeping state information for employment with an adaptive alarm.

While sleep is a highly discussed and researched topic in the U.S. and other Western cultures, and eight hours of sleep nightly is a commonly cited recommendation, some people choose to sleep less and other people due to employment, family, or other concerns, simply cannot take such a long duration to sleep. Consequently, for many people, it is important to take maximum advantage of what years of research has defined as discernable sleep cycles of nightly sleep.

In recent years, studies have shown that to wake up refreshed, even after a minimal sleep session, it is advantageous to actually awaken during a period of a lightest sleep cycle. Research has shown, that awaking during this time period in a period of a light sleep cycle helps the sleeper to have more energy and be more alert on waking and during the day.

When a human sleeps, the body and their mind experience different levels of sleep throughout the night or their duration of sleep. Conventionally, research has shown that each person sleeps through a plurality of different sleep cycles which include REM sleep, where the sleeper moves little if at all but experiences a Rapid Eye Movement (REM), and more rapid breathing, and sequentially more alert states where the body will move, as will the head, but there is no rapid eye movement. REM sleep occurs at approximately ninety minute intervals. REM sleep is characterized by high frequency EEG activity, bursts of rapid eye movements, REM sleep muscle atonia, and heightened autonomic activity. Most people move through these different cycles sequentially about four to five times a night.

A typical night's sleep for a normal person quickly transitions to a sleep stage known as slow wave sleep (SWS) characterized by low frequency electroencephalogram (EEG) activity. At approximately ninety minute intervals, sleep lightens and a sleep state known as rapid eye movement (REM) sleep is initiated. REM sleep is characterized by high frequency EEG activity, bursts of rapid eye movements, skeletal muscle atonia, and heightened autonomic activity.

In light stages of sleep, the brain begins the process of shutting down to a deeper sleep. Most sleepers refer to this as a feeling of dozing-off. During this light sleep cycle, the sleeper may be easily awakened by noise or even thoughts. This first stage is usually followed by a second stage of slightly deeper sleep where the body and head of the sleeper still moves on the bed and pillow, but the brain starts to move toward a deep sleep cycle.

During these deeper sleep cycles, body motion and activity moves to a second or lower state. Body internal activity lowers along with the lessening of motion while conversely activity in the brain is very high.

In a third cycle of sleep, REM sleep occurs for about 60-90 minutes. This is the deepest stage of sleep and the body is substantially paralyzed from motion by the brain while conversely the eyes of the sleeper will move rapidly. Breathing rates tend to increase concurrently. It is in this REM stage that sleepers dream and it can be very hard to wake a sleeper in REM, and if they are awakened, they can frequently feel disoriented upon awakening, and will often feel tired during the day thereafter.

As such, it would be advantageous to provide a method and apparatus allowing a sleeping user, unconnected physically by wires, as is the case with conventional system, to be able to monitor and store to electronic memory of a controller having a computing component, the sensory data concerning differing physiological aspects of their body during the begriming and light stages of sleep, as well as during REM sleep. Such a system by storing such sensor information in computer memory for subsequent analysis, would allow the user to ascertain what may be causing them not to fall asleep, by reviewing the electronic sensory data communicated to the controller concerning sound, motion, heart rate, position on the pillow, and other monitored conditions during light sleep.

Further, by tracking data concerning physiological aspects of the sleeper garnered from the pillow-positioned sensors, such a system can input multiple data streams from the multiple sensors, to memory in the controller, and ascertain a subsequent wake time to cause the user to be awakened during a calculated light sleep cycle, at a time proximate to the set alarm most likely to prevent them from being in a deep sleep. Such a system in acertaining the best awakening time, would employ the software to take into consideration the alarm time set by the user for awakening, and would employ captured sensory data to calculate the most appropriate time to wake the user with an alarm, prior to the user-set alarm, and thereby avoid the above noted problems caused by being awakened during REM sleep.

Such a system, employing software adapted to the task of tracking data from the various sensors, and running on a computing device in communication with data from appropriately placed sensors, should employ the software to intake the communicated electronic data concerning a plurality of body functions and position states of a sleeper, and thereby ascertain the current sleep state of the sleeper, before waking them.

Using a net or plurality of pillow positioned sensors, the system herein insures data is garnered concerning the appropriate physiological aspects of the sleeper, no matter the position of the sleeper on the pillow. The web of sensors in three different areas of the pillow, allows the system to continually ascertain the position of the sleeper, and no matter the position on the pillow, gather the necessary electronic data using strategically positioned sensors in each area.

Using the multiple sensors for each type of captured electronic data concerning physiological aspects of the user or sleeper, the device and method herein accomplishes this task with software configured to employ garnered information from data streams from each sensor, concerning a sleeper's current and past motion, current head position, sound, temperature, and duration since last motion, when their head is adjacent or in contact with any area of the pillow.

For example, a side sleeper will generally contact an off-center portion of the pillow with one side of their face, depending on which shoulder they have rolled in a contact with the mattress. Thus, a current position of a sleeper, either on one shoulder with the face on one side of the pillow or on the opposite shoulder, can be discerned by sensors in segments of the pillow, which can be confirmed by sound sensed, temperature sensed, proximity, and pressure sensors which ascertain head weight, and other sensors positioned within the regions of the pillow.

Besides these side positions, a third position for the head of most sleepers in contact with the pillow, communication of the rear of their head, which positions to a central area of the pillow when the user's back is in contact with the mattress along with both shoulders being proximate to the mattress. As with the side positions of the face, the duration of the sleeper on their back, can be roughly calculated by the duration of contact of the middle of the back of their head, with a central portion of the pillow using one or a plurality of the noted sensors above.

Since a major element of REM sleep is non-movement or lack of body motion of the sleeper, and a period of rapid eye moment, the number and duration of REM periods of sleep can be determined by software running on a computing device using communicated data from the sensors sensing positioning, and motion, of the head on the pillow which when communicated to the software on a computing device will keep track of movements or motion, and the frequency, and the times of non-motion. Employing this data concerning motion the software can determine times of minimal or non motion in a motion value, to help ascertain REM sleep, especially when combined with other sensory data.

Additionally, rapid breathing can be discerned by microphones, and temperature variations of the head can be detected by thermometers both of which will communicate data concerning breathing and temperature to the computing device for tracking and use by the software employed to track sleep patterns and determine REM periods, and periods of light sleep.

Further, a sensor such as an accelerometer can discern the speed of and actual motion, by the movement of the pillow, and whether the sleeper is moving or simply, for example, twitching. The data streams electronically communicated concurrently from all the sensors, to a computing device such as a smart phone or pad computer, can be stored in electronic memory and processed in real time, or subsequently, by software running thereon adapted to use the data streams of information, to determine current and previous motion periods and the cycle of sleep the sleeper is in currently.

Based on such, software running on the computer can, using statistical norms, or more preferably, an onboard database held in electronic memory of prior sleep of the user, and calculate a most probable light sleep cycle, which will be proximate to a requested wake-up time. Once a wake time is ascertained for waking the user by the software, the system can employ a connected alarm means to begin waking the user such as a table or room lamp light, which becomes progressively brighter to mimic the sun, and after a period of light, the employment of a loudspeaker to produce sound which becomes progressively louder.

As such, employing the data streams electronically communicated, from the plurality of pillow engaged sensors, located in multiple zones on the pillow to insure continual communication, the sleeping user can employ the device herein as a means to track their time falling asleep to ascertain possible problems, and as an adaptive alarm to wake them at a calculated wake time, when they are not in deep sleep or REM sleep. Further, the system can do so gradually with light and/or sound to avoid startling the sleeper as with many alarms. This will result in a more energized and alert user for the coming day.

It is thus an object of this invention to provide an improved pillow which includes a plurality of sensing components, located in a plurality of zones on a pillow, which will provide electronic data to software running on a computer configured to use the data to monitor the user's sleep cycles.

It is another object of this invention to provide such a device which can employ the garnered data from the sensors with software adapted to the task of calculating a most likely non-REM sleep duration, and then providing a wake time to activate an alarm for the user when they are not in REM or deep sleep.

A further object of the invention is the provision of such a device which employs software configured to use sound and other physiological data from sensors, to discern if users are suffering from sleep apnea or other breathing or snoring difficulties during sleep.

These together with other objects and advantages which become subsequently apparent reside in the details of the configuration and method of operation of the device and method herein, as more fully hereinafter described and claimed, reference being had to the accompanying drawings forming a part thereof, wherein like numerals refer to like parts throughout.

### BRIEF DESCRIPTION OF DRAWING FIGURES

FIG. 1 depicts a top view of one mode of placement of sensors in three segments of a pillow.

FIG. 2 depicts a side view showing the sensors and possible extension of soft sensors, if needed, closer to the top surface of the pillow.

FIG. 3 depicts the exterior of a pillow with the sensor web of FIG. 1 operatively positioned and with a cover on the pillow showing the normal appearance.

FIG. 4 is a graphic depiction of communication wirelessly or wired between the transceiver and controller having a computing device and operative software, and an alarm.

### DETAILED DESCRIPTION OF THE PREFERRED EMBODIMENTS OF THE INVENTION

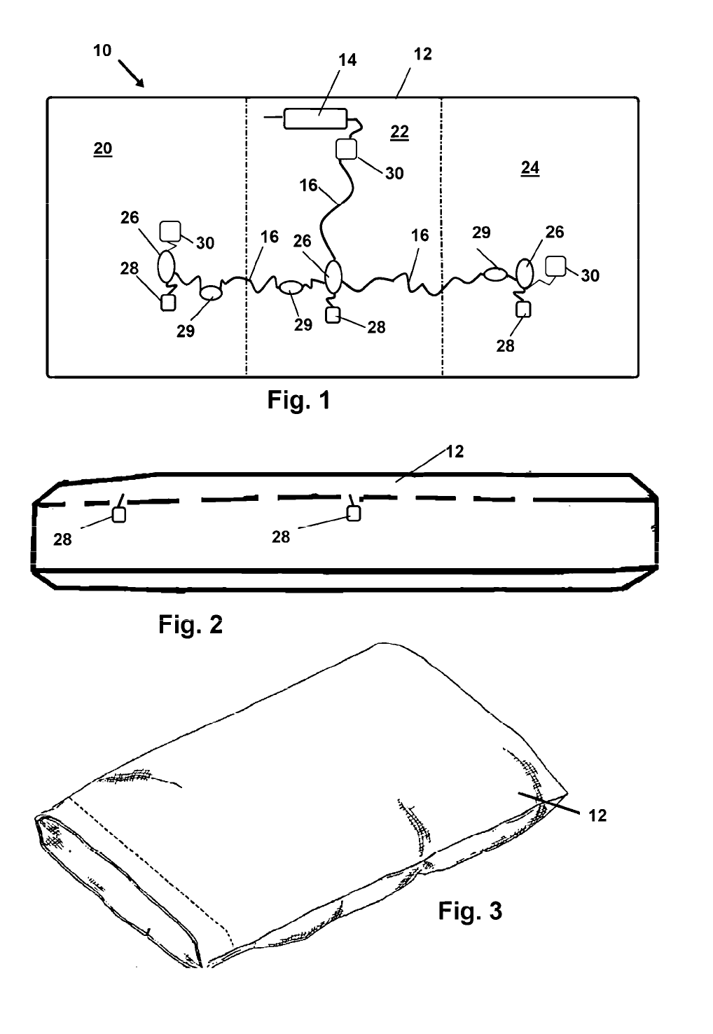

Referring now to the drawings of FIGS. 1-4, wherein like numerals refer to like parts, there is seen in FIG. 1 a top view of one mode of the device 10 wherein a pillow 12 has a plurality of sensors operatively positioned within the body of the pillow, to sense user motion which moves the pillow, temperature, noise, and bodily functions. Such sensors can be one or a plurality of sensors from a group including sound sensors or microphones 39, temperature sensors 29, proximity sensors, and weight or compression sensors 28, and motion sensors 26 such as accelerometers.

As shown in one preferred mode of the device 10 and system herein, a plurality of pillow-positioned sensors can be in operative communication with a computing device directly or using a wireless transceiver 14 which will communicate data streams from the sensors, to a controller 15 having a computing device therein and electronic memory for running software, which is dedicated to the system herein, or employable by engagement to the sensors in a wired, or wireless communication, such as a smart phone or pad computer.

The controller 15 in any case will have a computing device with electronic memory for storage of the individual data streams from each sensor, in each individual area of the pillow 12. The computing device of the controller 15 will have software in electronic memory thereon, configured to employ the individual data streams, from the respective individual sensors, in each area of the pillow 12, which provide information to ascertain motion, user position and durations thereof, motion, temperature in each area of the pillow 12, sounds made by the user such as during respiration, respiration rate from such sounds, and employ an algorithm or a software to make a calculation of the current sleep level value, which relates to the user's current state in their sleep cycle. The algorithm may simply sum the ascertained respiration values and motion values and temperature values, may multiply them, or may employ weighted values of each in a final sum or product which would derive the sleep level value.

Two primary factors in determining the current sleep level value, have been found in experimentation to be based on a calculation using a current user motion value, and a respiration value based on respiration rate and patterns of sound in a signal pattern thereof during the duration of time of sampling. By current motion value and respiration value is meant a respective motion value ascertained from data communicated from the respective motion sensors 26 and the signal pattern from sounds communicated from microphones 30, over a time duration such as 1 to 10 minutes. A value such as a temperature value would be ascertained from electronic data from the temperature sensors 29 concurrently communicated during the same time duration as other sensors. The ascertained motion value and respiration value and temperature value, or other value from sensors obtained concurrently during any one duration of time of sampling may also be employed in making the calculation for a current sleep level value.

It is well known that during REM sleep, body movements virtually cease or significantly lessen as opposed to the body movements when in light sleep where the user turns over and moves arms and legs causing movement of the bed and pillow which may be sensed by an accelerometer or other motion sensor 26. Further it is well known that respiration rate during REM increases over that of regular sleep, and can also happen in a rhythm which is different than the essentially even rate of respiration during lighter sleep.

Using data communicated electronically from at least one microphone 30 from one of the segments of the pillow 12, a current respiration value can be calculated from the number and frequency of breathing sounds, over a time duration noted above.

Also, using a motion sensor 26, such as an accelerometer, or the weight or compression sensor 28 in each area of the pillow 12, over the same duration of time as that of the microphone 30 or other sensors used in the calculation, a motion value may be ascertained by software running on the computer of the controller 15. For instance the number of times a weight or compression sensor 28 in each respective part of the pillow 12, shows more weight than the others, over the time duration, and the location of each, can show how many times the user's head moved during the time duration, which can be calculated as the motion value can be ascertained.

The respiration value may be aided by the software running on the computer of the controller 15, which may compare the frequency and tone levels of the sounds captured from the user's breathing, to a database in electronic memory of known tone levels and respiration frequency and rhythm related to known respiration values, relating to light sleep, medium, sleep, and REM sleep. Comparing the respiration rate, tone, and frequency captured from the microphone 30s during the measurement time duration, to that of known respiration values for sleep levels, a current respiration value may be calculated.

Regarding the motion value, the software running on the computing device of the controller 15, can use the input data from the motion sensors 26 such as accelerometers in the pillow 12 sensing motion of the user over the time duration, and/or weight sensors 28 in each area of the pillow which sense head-weight in each area, for durations of time. Electronic data from an accelerometer can provide information relative to motion and speed of such motion over the time period. Electronic data from the weight sensors 28 allow calculation of head positions over the duration of time. Software for calculating a current motion value, can employ one or both inputs of data from the time duration, to calculate if the user is substantially not moving during the time duration, or moving many times, and using this data from motion, calculate the motion value for the duration of time of measurement. This motion value may be employed in the current sleep level value, or may be compared to a database of motion values related to sleep levels to ascertain a current motion value, which may be combined with the respiration value or any other value ascertained from sensors during the time duration of measurement, to calculate the current sleep level value.

In one preferred mode of the device, with the simplest operation, the current motion value and the current respiration value ascertained over a concurrent time duration, can be employed to calculate a sleep level to determine REM sleep or non REM sleep. A simple calculation using a sum of the two values, can arrive at the current sleep level value. This calculated sleep level value, can be used in a comparison to a scale of known such sleep level values held in memory, determined from a sum of respiration values and motion values, to determine the current sleep level of the user being REM or non REM. Or alternatively, in a simple calculation the sum itself may be the current sleep level value. If other sensors are employed with measurements of other physiological values during the concurrent time duration, they can be included in the sum, or other calculation to determine the current sleep level value.

This sleep level value may then be correlated with a sleep level value scale, held in electronic memory, where a particular calculated sleep level value or one within a range of sleep level values are known to correlate to a user in REM sleep, and, where known sleep level values outside that range correlate to non REM sleep. Using the current calculated sleep level value compared to those of the scale in electronic memory, it is thus determinable if the user is in REM sleep, or not. Because REM sleep occurs in predictable periods, which can over time, using captured data from the user as to how often and the time durations of the user's REM sleep durations, be very predictable.

By using the current ascertained sleep level value, and the time duration from the last ascertained sleep level value calculated to show REM sleep, the software running on the computing device of the controller 15, can calculate when a next REM sleep duration will occur. If the user has set a wake time which the software running on the computing device calculates will occur during the next calculated REM sleep duration, an alarm will be signaled to wake the user during the lighter sleep duration prior to the next REM period. If the next REM sleep duration is calculated to occur after the user input wake time, then the alarm will be allowed to wake the user at the chosen time.

Shown in FIG. 1 is a wiring buss 16 placing all of the individual sensors in electronic communication with a transceiver 14 for communicating data to the controller 15 having onboard electronic memory and a computing device running software configured to ascertain the respiration value and motion value, and any other values employed by the software configured to calculate a current sleep level value.

The buss 16 may be individual direct wiring of each sensor to the transceiver 14, or it may use the same wires to multiplex signals from each and communicate data from each respective sensor to the transceiver 14 or computing device. A battery 17 or other electrical power is positioned on or in communication with the transceiver 14 to communicate power to the various sensors employed, along wires of the buss 16.

Preferably, each individual sensor also has an identifier, associating communicated data from each respective sensor with its individual identifier. This identifier may be ascertained by the wiring between each sensor along the buss 16 to the transceiver 14, or each sensor may have a built in electronic identifier which is transmitted at periods during transmission of the data ascertained by that sensor.

In this fashion, the location of each sensor ascertained by a respective identifier, can be communicated to the controller 15 whereby the software running on the computing device of the controller 15, can use that position or location of each sensor on the pillow 12, to ascertain from which position on the pillow 12, the individual data stream is being sent.

This is important in the case of weight sensors 28, and motion sensors 26, in each area of the pillow 12, to determine the position of the user, based on time and the strength of the signal sent. For example a weight signal from a sensor in a location on the pillow 12, will be higher when such sensors are adjacent or under the user's head, whereby signals form weight sensors in known positions elsewhere on the pillow 12, will show a lighter weight signal.

Further the duration of non motion, or movement, during a time duration, can be ascertained, since higher weight sensed on each known weight sensor 28 in its known position on the pillow 12, will show where the head of the user has moved, and can be used to calculate motion and duration of non motion from ascertaining the positioning of the head over the time duration of sensor measurement used in a motion value calculation.

As shown herein, in a particularly preferred mode, the pillow 12 is divided into three segments, however two segments may also be employed where the pillow 12 would be divided in half sections instead of three segments. An end segment 20, a centrally located segment 22, and an opposing end segment 24, each have at least one, and preferably a plurality of the same sensors, located therein and operatively engaged with the wiring buss 16 to for electrical power and to communicate perceived electronic data from each sensor to the transceiver 14.

Particularly preferred, the wiring employed for the buss 16 is shown communicating between sensors and the transceiver 14, in a zig-zag configuration. This is most important because it was found during experimentation, that linear wired connections, without the zig-zag path, tended to break over time causing cessation of data from one or more sensors. However the zig-zag path was found as a solution to the problem in that it which allows extra wiring length to accommodate deformation of the pillow 12 by the user's head. This solution maintains the wiring for data and power in the buss 16 operative by allowing the wires to stretch and compress as needed depending on the position and movement of the user's head.

Shown positioned in each segment are a motion sensor 26 and a compression or weight sensor 28. Although, other or more sensors may be included. The motion sensor 26 such as an accelerometer can determine motion and speed of movement in each of the segments of a head on the pillow 12. A weight scale or compression sensor 28 in each segment can determine the head placement on the pillow 12 and to which segment the user's head is located which correlates with the position of the body being on a right or left shoulder or the user's back.

Also included can be a microphones 30 which may be tuned using software or electronic filters or physical configuration to be especially sensitive to the sound frequencies of breathing. Since breathing rate also slightly raises and lowers the body, partially due to lung expansion, the weight or compression or weight sensors 28 in combination with data from the microphones 30, may be employed in combination, to better determine breathing rates when determining sleep cycles as well as to track or detect snoring and/or sleep apnea.

Employing software running in electronic memory of the computing device of the controller 15 herein, adapted to intake the electronic data from, from the multiple microphones 30 in multiple pillow areas 20, 22, and 24, the system herein can function to ascertain the onset of sleep apnea and track it or even wake the sleeper if necessary. The controller 15 employing a computing device therein can employ software running in electronic memory, adapted ascertain a signal pattern of the user's breathing sounds captured by the microphones 30 or the duration of time of sensor sampling. The captures signal pattern can be compared to signal patterns in a database which are associated with a breathing pattern of a person which occurs at the onset of an obstructive sleep apnea event. Such breathing patterns are well known, and a database of signal patterns of such breathing patterns, which are associated with the onset of sleep apnea, can be held in memory whereby the signal pattern from the microphones 30 for the duration of time of sampling can be compared to the signal patterns of the database, and sleep apnea onset can be ascertained if a substantial match is found.

Additionally, temperature sensors 29 may be placed in one position or all three segments 20, 22, 24, of the pillow 12 which will discern a temperature in that segment, which will be affected by the position of the user's head therein, or elsewhere. The temperature sensors 29 in positions on the pillow not covered by the user's head, will tend communicate a lower temperature than the temperature sensors 29 under or adjacent the head of the user on the pillow. The system herein can employ the multiple signals from the temperatures sensors 29, to provide another data stream allowing the software to determine positions on the pillow which are warmer and cooler, and to discern both head and body position. The higher temperature from the temperature sensors 29 under or adjacent the head of the user, during the duration of time of sampling, can be used as a temperature value, as another factor in the calculation of the current sleep level value used to determine the REM or non-REM sleep level of the user.

Thus, the system and device 10, during the same time duration, using the sensor data communicated electronically in streams from each respective sensor, in each of the regions, either in a wired connection or using the transceiver 14, to a computing device such as in the controller 15, will employ onboard software running in computer memory, to employ the data to discern current a sleep level value, of the user. Based on the time the user has been asleep or the first determined REM sleep cycle, and the determined current sleep level value, the a calculation can be made of when to wake the user based on whether a user is in a REM cycle, or has traversed to a light sleep cycle, based on the duration between REM sleep cycles known from previous determinations held in computer memory, and the duration in-between such REM sleep cycles for the user. Essentially software running on the computing device would be configured to learn the user sleep pattern timing over a duration of time such as a week, and use that data to ascertain the timing of REM sleep patterns and those in-between. Based on the first ascertained REM sleep pattern in any given sleep cycle, the subsequent REM sleep cycles may then be estimated for time occurrence, and used to determine a calculated wake time as noted herein.

This calculated wake up time may also be influenced by a user input of a desired time or time range to awaken, whereby the software employing the data streams from the sensors, will calculate a current sleep level value of the user, and ascertain when they subsequently will be in a light sleep cycle, closest to the chosen waking time to awaken the user based on the time from the first REM cycle of the evening, and previous learned data of the user timing of such for a given sleep cycle. At this calculated awakening time an alarm 35 will be activated to awaken the user.

As noted above, one manner of determining the current sleep level value will be to add the current motion value and respiration value to calculate a current sleep level value which can be predetermined to relate to REM cycle sleep if the sleep level value falls into a range on a scale, and non REM if it falls outside the range. Alternatively a current temperature value can be included in the sum determining a sleep level value. As a further alterative the sum of values may determine a calculated sleep level value which can be compared by software running on the computing device, with a scale of such sleep level values held in a database in memory which are known to relate to REM cycles where REM sleep determined, if the calculated sleep level value falls in a range thereof in the database which is predetermined to indicate REM cycle sleep.

As noted FIG. 2 depicts a cut away view showing the sensors. In order to keep the top layer of the pillow 12 soft, possible extensions which are flexible and soft or soft sensors, if needed, may be positioned closer to the top surface of the pillow 12.

Because aesthetics are so important to many users, FIG. 3 depicts the fact that the exterior of a pillow 12 with the sensor web of FIG. 1 operatively positioned, and with a cover on the pillow 12 will be normal in appearance and overall function.

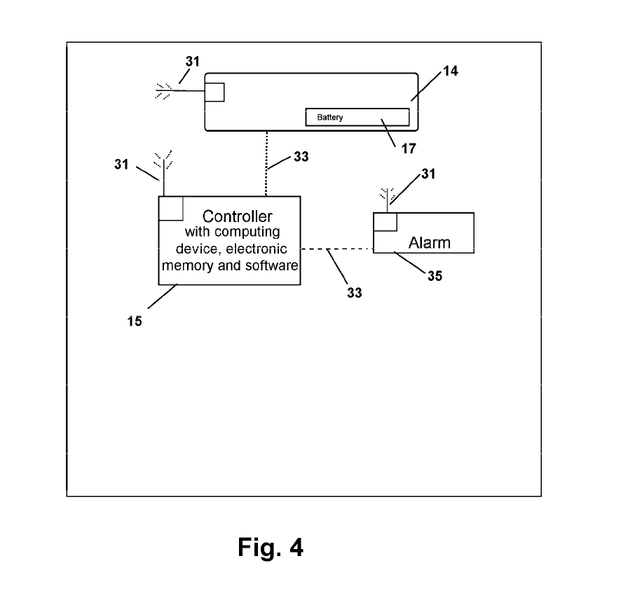

Finally, FIG. 4 shows a graphic depiction of the electronic communication, wirelessly 31 or wired 33 between the transceiver 14 receiving the data streams from the sensors, and controller 15 having a computing device with electronic memory and operative software thereon, and an alarm 35. A battery 17 or another source of power is shown powering the transceiver 14 and would also provide electric power to the sensors herein via operative wires included in the buss 16. The components as shown may communicate wirelessly 31 such as with BLUETOOTH or Wifi which would work especially well with smartphones and pad computers if they function as the controller 15, and host the software in memory thereon as well as any databases for comparison of captured sensor data to known norms or user norms, to calculate the current sleep value of the user in the aforementioned fashion, and activate the alarm 35 at the calculated wake up time as noted if needed.

While all of the fundamental characteristics and features of the disclosed sleep sensing pillow device and method have been shown and described herein, with reference to particular embodiments thereof, a latitude of modification, various changes and substitutions are intended in the foregoing disclosure and it will be apparent that in some instances, some features of the invention may be employed without a corresponding use of other features without departing from the scope of the invention as set forth. It should also be understood that various substitutions, modifications, and variations may be made by those skilled in the art without departing from the spirit or scope of the invention. Consequently, all such modifications and variations and substitutions, as would occur to those skilled in the art, are considered included within the scope of the invention as defined by the following claims.

What is claimed is:

1. A pillow apparatus comprising:
a motion sensor positioned within said pillow, said motion sensor generating an electronic motion signal from sensed motion on said pillow;
a sound sensor positioned within said pillow, said sound sensor generating an electronic sound signal from respiration sounds of a user of said pillow;
a controller having a computing component and electronic memory;
said controller connected to receive said electronic sound signal and said electronic motion signal;
software running in said electronic memory of said computing component, said software configured in a first calculation, ascertaining a motion value based on said motion signal received during a duration of time, and a respiration value based on said electronic sound signal received concurrently during said duration of time; and
said software in a second calculation, determining a sleep level value of said user, correlating to REM sleep, using a combination of said motion value and said respiration value, whereby during an entire sleep cycle of said user, said sleep level value is employable to determine periods of REM sleep and periods of non-REM sleep.

2. The pillow apparatus of claim 1, additionally comprising:
said software in a third calculation, estimating a start time and duration of a subsequent period of REM sleep of said user subsequent to an end of said duration of time, based on said sleep level value of said user and an elapsed time since an end of a previously determined period of REM sleep of said user.

3. The pillow apparatus of claim 2, additionally comprising:
said controller is in operative communication with an alarm, said alarm having a timed activation to wake said user, at a user determined time;
said alarm configured for an override of said timed activation by an activation signal communicated from said controller; and
said controller configured to communicate a said activation signal earlier than said user determined time, to activate said alarm to wake said user, if said user determined time falls within said duration of a subsequent period of REM sleep.

4. The pillow apparatus of claim 1 wherein said sleep level value is determined by the sum of said motion value and said respiration value.

5. The pillow apparatus of claim 2 wherein said sleep level value is determined by the sum of said motion value and said respiration value.

6. The pillow apparatus of claim 3 wherein said sleep level value is determined by the sum of said motion value and said respiration value.

7. The pillow apparatus of claim 1 wherein said first calculation also includes ascertaining a temperature value based on a temperature signal received from a temperature sensor engaged with said pillow during a duration of time and said software in said second calculation, determining a sleep level value of said user, correlating to REM sleep, includes using a combination of said motion value and said respiration value and said temperature value.

8. The pillow apparatus of claim 3 wherein said first calculation also includes ascertaining a temperature value based on a temperature signal received from a temperature sensor engaged with said pillow during a duration of time and said software in said second calculation, determining a sleep level value of said user, correlating to REM sleep, includes using a combination of said motion value and said respiration value and said temperature value.

9. The pillow apparatus of claim 8 wherein said sleep level value is determined by a sum of said motion value and said respiration value and said temperature value.

10. The pillow apparatus of claim 3 wherein said sleep level value in said second calculation is determined by an algorithm using one or a combination of calculations from a group including, a sum the ascertained respiration values and motion values, weighted values of each of said respiration values and said motion values, in determining a final sum or product equaling said sleep level value.
